(19) United States
(12) Patent Application Publication
(10) Pub. No.: US 2026/0051405 A1
(43) Pub. Date: Feb. 19, 2026

(54) ARTIFICIAL INTELLIGENCE-BASED PERSONALIZED CARE DELIVERY SYSTEMS AND METHODS

(71) Applicant: Centene Corporation, Saint Louis, MO (US)

(72) Inventors: Hugh H. Ryan, Lee's Summit, MO (US); Barrett C. Foat, Ballwin, MO (US); Lori A. Howard, Leawood, KS (US)

(21) Appl. No.: 19/303,006

(22) Filed: Aug. 18, 2025

Related U.S. Application Data
(60) Provisional application No. 63/684,580, filed on Aug. 19, 2024.

(51) Int. Cl. G16H 50/20; G16H 10/60; G16H 40/20; G16H 80/00

(52) U.S. Cl. CPC G16H 50/20; G16H 10/60; G16H 40/20; G16H 80/00

(57) ABSTRACT

A personalized health plan (PHP) computing system is described. The PHP computing system is configured to receive member health data associated with a member and apply the member health data to an observation model trained to identify health conditions of the member based upon historical health data. The PHP computing system is also configured to receive a health conditions output from the observation model and apply the health conditions output to one or more service models wherein the one or more service models are trained to generate treatment plans associated with care for different health conditions. The PHP computing system is further configured to receive a service response output from the service model, cause a first notification for a recommended health appointment to be transmitted to a care provider computing device, and cause a second notification for the recommended health appointment to be transmitted to a member computing device.

### CROSS REFERENCE TO RELATED APPLICATION

This application claims the benefit of priority to U.S. Provisional Patent Application No. 63/684,580, filed Aug. 19, 2024, which is hereby incorporated by reference herein in its entirety.

### FIELD OF THE DISCLOSURE

The field of this disclosure relates generally to artificial intelligence (AI) and personalized care delivery and, more specifically, to AI-based computer systems and methods for generating a personalized healthcare plan for each of a plurality of care members of the plan and/or one or more healthcare providers of the plan, and for delivering the healthcare services to the members while adjusting the plan in real-time as health conditions change for the members.

### BACKGROUND

In the data industry, data producers may play a critical role in providing data to data consumers that enable the data consumers to make informed decisions based upon analysis of the data provided by the data producers. Data producers may collect and generate data, for example, by capturing user interactions, sensor data, and/or from other external or specific resources or processes. The data may then be provided to data consumers upon receiving a request for data access from the potential data consumers.

Oftentimes, requests for data may be made via email, and access for data may be granted or denied via email. In many of these cases, the mechanisms that may be required to ensure data integrity, data security, and data reliability may be missing or not a concern for either the data producers or the data consumers.

Additionally, the types of data that may be available from such data producers to such data consumers and the different use cases of such data may not necessarily be clear to the data producers or the data consumers. Therefore, a substantial amount of human resources and time may be required to ensure that the data producers have the correct type of data required by the data consumer for the specific use case of the data consumer. Similarly, a substantial amount of human resources and time may be required to ensure that the data being provided by the data producers is being used by the data consumer in a manner intended for by the data producer.

In many cases, data needs to be labeled or classified in order to perform further analysis, including in those cases where AI or ML tools are applied. For example, in the healthcare industry, there are many variables that need to be considered for automatically classifying data. When classifying data, certain factors may be known and certain factors may be unknown. Different entities may provide different portions of the data and/or the data may be in different formats. Data that is incorrectly classified typically results in inaccurate outputs.

As an example, in some cases, some known healthcare platforms include a care team, a healthcare provider, a member or patient, and a community. The care team may include a coordinated group of healthcare professionals and support staff dedicated to delivering comprehensive, patient-centered care. A member refers to the patient receiving care within the framework of the healthcare system. The community includes the broader social and environmental context in which the member resides.

The known methodology for providing care within the framework of a care team, provider, member, and community typically involves a structured, manual approach to healthcare delivery. Initially, patients, referred to as members, are enrolled by gathering essential health information. The member may then be provided a multidisciplinary care team, led by a primary care provider, to coordinate the development of a generalized care plan for the members in the group. The team may manually develop treatment protocols, medication management, and follow-up schedules for the members in the group. Care delivery may be executed according to this plan, with providers conducting periodic check-ups, treatments, and health education.

Unfortunately, these known care plans are disjointed and generalized, leading to significant inefficiencies in data gathering, data transfer, and communications between the parties involved. Conventional methodologies often result in critical health information being fragmented across various platforms and providers. This fragmentation causes delays and errors in information exchange, with essential data sometimes missing or not reaching the appropriate members of the care team in a timely manner. Additionally, utilization management (UM) methods, while aimed at cost containment, are often too expensive and burdensome to implement effectively. Case management (CM) methods tend to result in the overuse of resources by a small group of members, thereby restricting the capabilities to provide adequate care for the broader member population. These limitations further add to the challenges of disjointed information flow, hampering the coordination and effectiveness of patient care. Consequently, there is a need for a more integrated and seamless health plan platform within the healthcare framework to provide specialized and personalized care for each member.

Other limitations in these known care systems include an inability to deliver the healthcare treatments or other health care related services that may be prescribed for a member. Moreover, these known systems are unable to predict the services that may be required in the future for the member based on past treatments, and then deliver those services. In other words, a treatment may get prescribed for a member, but the system is not setup to ensure that the treatment is delivered to the member or, if it is delivered, these known systems are unable to be updated with the delivered treatment and the result thereof. These limitations result in substantial issues for the members with a lack of proper treatment, lack of informed decisions, and increased costs and additional support staff.

Other advantages from this improved system will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION

In one aspect, a personalized health plan (PHP) computing system including at least one processor in communication with at least one memory is described. The at least one processor is configured to receive member health data associated with a member from at least one of a database or a member computing device and apply the member health data to an observation model wherein the observation model is trained to identify health conditions of the member based upon historical health data. The at least one processor is also configured to receive a health conditions output from the observation model, the health conditions output including one or more health conditions associated with the member based upon the member health data, and apply the health conditions output to one or more service models wherein the one or more service models are trained to generate treatment plans associated with care for different health conditions. The at least one processor is further configured to receive a service response output from the service model, the service response output including a recommended treatment plan including a recommended health appointment for the member based upon a health condition of the one or more health conditions, cause a first notification for the recommended health appointment to be transmitted to a care provider computing device associated with a care provider to conduct the recommended health appointment for the member, and cause a second notification for the recommended health appointment to be transmitted to the member computing device to notify the member of the recommended health appointment with the care provider.

In another aspect, at least one non-transitory computer-readable storage medium with instructions stored thereon for personal health planning is described. The instructions, when executed by at least one processor, cause the at least one processor to receive member health data associated with a member from at least one of a database or a member computing device and apply the member health data to an observation model wherein the observation model is trained to identify health conditions of the member based upon historical health data. The instructions also cause the at least one processor to receive a health conditions output from the observation model, the health conditions output including one or more health conditions associated with the member based upon the member health data, and apply the health conditions output to one or more service models wherein the one or more service models are trained to generate treatment plans associated with care for different health conditions. The instructions further cause the at least one processor to receive a service response output from the service model, the service response output including a recommended treatment plan including a recommended health appointment for the member based upon a health condition of the one or more health conditions, cause a first notification for the recommended health appointment to be transmitted to a care provider computing device associated with a care provider to conduct the recommended health appointment for the member, and cause a second notification for the recommended health appointment to be transmitted to the member computing device to notify the member of the recommended health appointment with the care provider.

In another aspect, a computer-implemented method for personalized health planning is described. The computer-implemented method is implemented by at least one processor and at least one memory. The computer-implemented method includes receiving member health data associated with a member from at least one of a database or a member computing device and applying the member health data to an observation model wherein the observation model is trained to identify health conditions of the member based upon historical health data. The computer-implemented method also includes receiving a health conditions output from the observation model, the health conditions output including one or more health conditions associated with the member based upon the member health data, and applying the health conditions output to one or more service models wherein the one or more service models are trained to generate treatment plans associated with care for different health conditions. The computer-implemented method further includes receiving a service response output from the service model, the service response output including a recommended treatment plan including a recommended health appointment for the member based upon a health condition of the one or more health conditions, causing a first notification for the recommended health appointment to be transmitted to a care provider computing device associated with a care provider to conduct the recommended health appointment for the member, and causing a second notification for the recommended health appointment to be transmitted to the member computing device to notify the member of the recommended health appointment with the care provider.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

The exemplary embodiments are shown in the drawing arrangements which are presently discussed. However, it should be understood that the present embodiments are not limited to the precise arrangements and/or instrumentalities shown herein.

FIG. 1 illustrates an example flow diagram implemented by a personalized health plan (PHP) system, in accordance with the present disclosure.

FIG. 2 illustrates an example flow diagram of a coordination between a healthcare provider and a member as implemented by the PHP system, in accordance with the present disclosure.

FIG. 3 illustrates an example framework of an observation model of the PHP system, in in accordance with the present disclosure.

FIG. 4 illustrates an example flow diagram implemented by the observation model of the PHP system, in accordance with the present disclosure.

FIG. 5 illustrates an example framework of a service model of the PHP system, in accordance with the present disclosure.

FIG. 6 illustrates an example flow diagram implemented by the service model of the PHP system, in accordance with the present disclosure.

FIG. 7 illustrates an example flow diagram implemented by an assurance model of the PHP system, in accordance with the present disclosure.

FIG. 8 illustrates an example flow diagram implemented by a transition model of the PHP system, in accordance with the present disclosure.

FIG. 9 illustrates an example embodiment of a dashboard generated by the PHP system that is displayable on a computing device, in accordance with the present disclosure.

FIG. 10 illustrates an example flow diagram of a notification process implemented by the PHP system, in accordance with the present disclosure.

FIG. 11 illustrates an example embodiment of a deployment of the PHP system, in accordance with the present disclosure.

FIG. 12 illustrates an example flow diagram of distributing notifications for community rounds by the PHP system, in accordance with the present disclosure.

FIG. 13 illustrates an example flow diagram of distributing notifications for member programs by the PHP system, in accordance with the present disclosure.

FIG. 14 illustrates an example flow diagram of distributing notifications for outliers of a by cohort the PHP system, in accordance with the present disclosure.

FIG. 15 illustrates an example implementation of the PHP system, in accordance with the present disclosure.

FIG. 16 illustrates an additional exemplary implementation of the PHP system, in accordance with the present disclosure.

FIG. 17 illustrates an example embodiment of a server system, in accordance with the present disclosure.

FIG. 18 illustrates an example method 1800 for personalized health planning, in accordance with the present disclosure.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

The following detailed description illustrates embodiments of the disclosure by way of example and not by way of limitation. The description enables one skilled in the art to make and use the disclosure. It also describes several embodiments, adaptations, variations, alternatives, and uses of the disclosure, including what is presently believed to be the best mode of carrying out the disclosure.

The present embodiments may relate to, inter alia, systems and methods for generating a personalized health plan (PHP), more particularly, to an artificial intelligence (AI)-based computer system and method for generating a personalized managed care plan and providing healthcare services for members (e.g., patients) within a healthcare framework. The systems and methods described herein are implemented using a PHP system (e.g., a PHP computer platform). The PHP system is a managed care tool that ingests data member data including the members' health history, current family, home and work situation data, environmental data, and any other data related to or impacting on the health and care delivery of the members. The PHP system processes member data to populate recommendations into care team workflows. The PHP system centralizes all member data to provide real-time, frictionless episodes of care across providers for the members. The PHP system is applicable across all conditions, venues, and care events resulting from centralizing all of the member data. In this way, the PHP system follows the member across all episodes of care to change and adapt in real-time along with the member as additional information associated with the member is provided to the PHP system.

The PHP system enables a multidisciplinary approach to member and community focused managed care. To provide the managed care, the PHP system leverages machine learning (ML) and AI models to allow for care in parallel between the PHP system and care managers. The PHP system provides access to AI and ML tools to facilitate healthcare services across users of the platform through cloud technologies and data transformation capabilities to provide the PHP system for each member. For example, the models included within the PHP system process member data including member health data, location data, language data, and resource usage data (sometimes collectively referred to as member data). The models also process manager data of managers administering the healthcare system including manager locations, languages, caseload, and capacity (sometimes collectively referred to as manager data). Using the processed member data and manager data, the models pair a member with a manager that aligns with their member data. In this way, the PHP system improves over the conventional care frameworks by including aspects of utilization management (UM) while expanding case management (CM) capabilities.

Further, the PHP system is configured to leverage the ML and AI models to support both the care team and the community to further serve the members associated with the PHP system. For example, the PHP system embeds best practices into the PHP system for each member. The best practices embedded with the system help guide the services provided to the member including health maintenance, chronic and complex conditions, acute events, and transitions in care. Further, the PHP system identifies and adjusts the care plan in real-time for member changes in venue, member conditions, social situations, regulatory requirements, and member goals.

The PHP system is further configured to provide network-care alignment across communities. In one exemplary embodiment, the PHP system is implemented on a computer device having one or more processors in communication with one or more memories that centralizes member data. The centralized data provides streamlined utilization for healthcare services, care navigation assistance, and identifying and utilizing opportunities for care identified by the PHP system. Centralizing the member data also supports the members care team, healthcare providers, and community. For example, the PHP system benefits the care team by identifying work items associated with the members, notifying the care team of events or tasks that are recommended based on the members individualized PHP, providing checklists for the care team to provide services to the member, and assisting with reviews and/or checkups. The PHP system also benefits providers by providing packaged approvals and leveraging shared decision making to provide care to the members. The PHP system also benefits the community by identifying and enrolling eligible members into campaigns and programs to provide improved healthcare service for the members and the communities.

In the example embodiment, a personalized health plan (PHP) computing system including at least one processor in communication with at least one memory is described. The at least one processor is configured to receive member health data associated with a member from at least one of a database or a member computing device and apply the member health data to an observation model wherein the observation model is trained to identify health conditions of the member based upon historical health data. The at least one processor is also configured to receive a health conditions output from the observation model, the health conditions output including one or more health conditions associated with the member based upon the member health data, and apply the health conditions output to one or more service models wherein the one or more service models are trained to generate treatment plans associated with care for different health conditions. The at least one processor is further configured to receive a service response output from the service model, the service response output including a recommended treatment plan including a recommended health appointment for the member based upon a health condition of the one or more health conditions, cause a first notification for the recommended health appointment to be transmitted to a care provider computing device associated with a care provider to conduct the recommended health appointment for the member, and cause a second notification for the recommended health appointment to be transmitted to the member computing device to notify the member of the recommended health appointment with the care provider.

In some embodiments, the one or more service models are further trained to prioritize health conditions based upon historical severities of the health conditions. In some embodiments, the one or more health conditions include a plurality of health conditions wherein the service response output further includes severities of the plurality of health conditions and wherein the at least one processor is further configured to cause the first notification and the second notification to be transmitted before other notifications associated with the plurality of health conditions based upon the health condition being a highest priority health condition. In some embodiments, the at least one processor is further configured to cause a third notification to be transmitted to the member computing device after causing the first notification and the second notification to be transmitted, the third notification associated with a next highest priority health condition of the plurality of health conditions and including a recommended treatment to address the next highest priority health condition.

In some embodiments, the at least one processor is further configured to receive updated member health data associated with the member from at least one of the database or the member computing device, the updated member health data including at least one update to the member health data, input the updated member health data to the observation model, receive an updated health conditions output from the observation model, input the updated health conditions output to the one or more service models, and receive an updated service response output from the one or more service models, the updated service response output including an updated recommended treatment plan including a plurality of treatment actions, each treatment action being assigned a priority level.

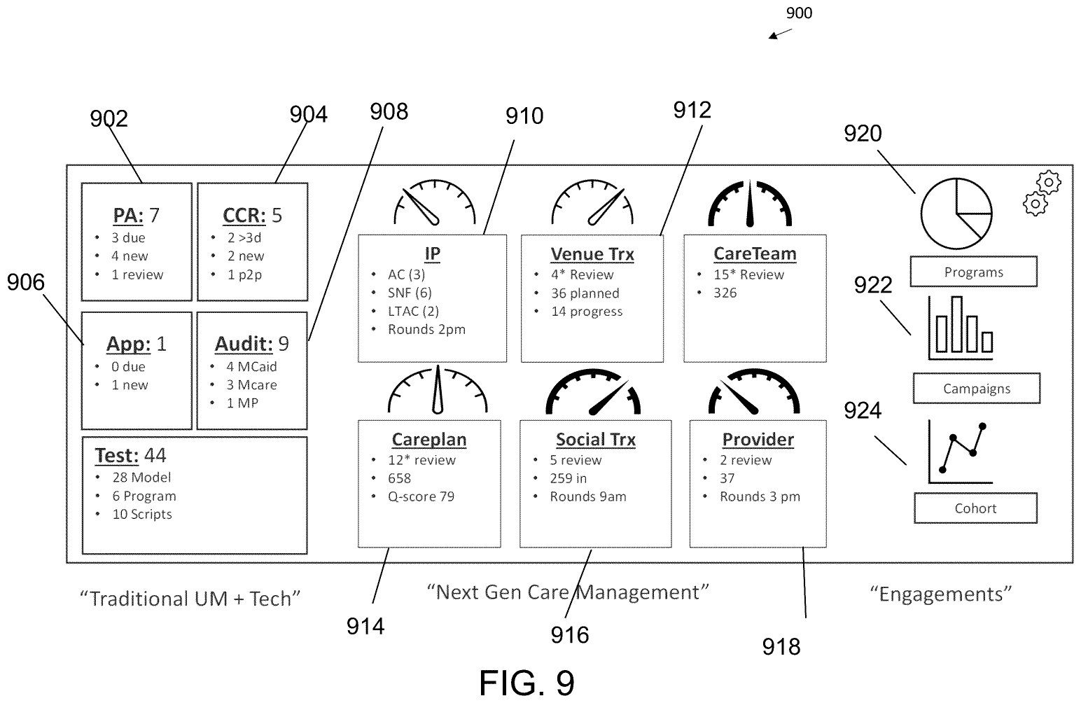

In some embodiments, the at least one processor is further configured to generate a graphical user interface (GUI) (e.g., see FIG. 9), the GUI including a first selector indicating that the recommended health appointment has been recommended to the care provider based upon the first notification and one or more second selectors associated with one or more additional appointments assigned to the care provider, wherein the GUI is updated in real time based upon outputs associated with the care provider from the one or more service models.

In some embodiments, the health conditions output includes duplicates of at least one health condition that were identified based upon different factors in the member health data wherein the at least one processor is further configured to apply the member health data to an assurance model, the assurance model trained to de-duplicate health data based upon historical duplicates identified in the historical health data, and receive an assurance output from the assurance model, the assurance output including an updated health conditions output including no duplicates of the at least one health condition wherein the health conditions output is updated with the updated health conditions output before the at least one processor applies the health conditions output to the one or more service models.

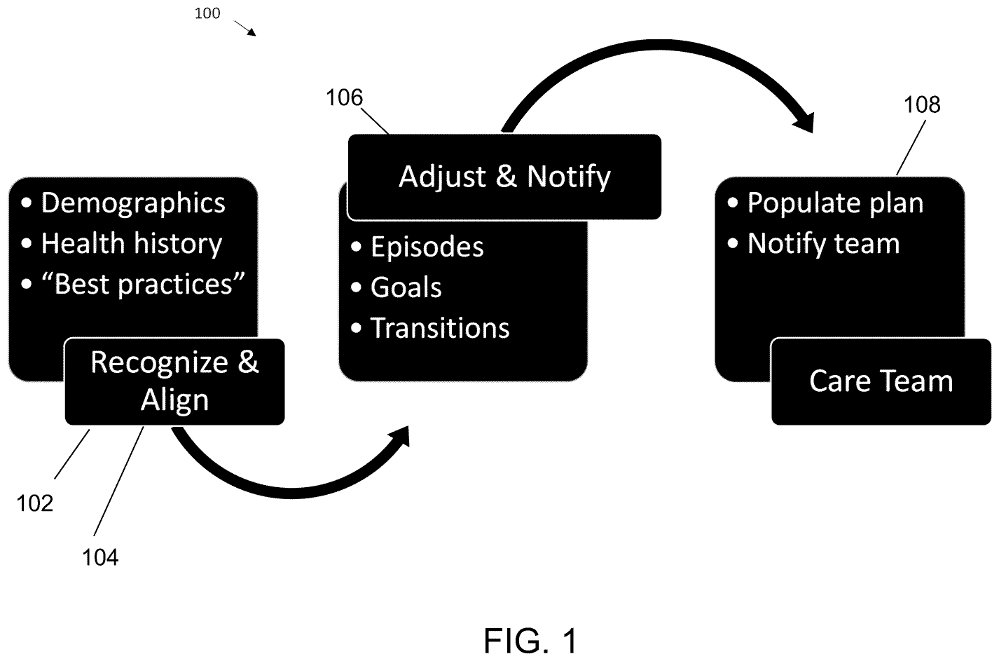

FIG. 1 illustrates an example flow diagram 100 implemented by a personalized health plan (PHP) system as described system as described herein. As shown in FIG. 1, the PHP system recognizes 102 (e.g., receives or identifies from one or more data sources) member data for a member such as demographic data, health history data, and best practice data associated with previous health plans and health outcomes. The PHP system aligns 104 the member data with the best practice data so that each member receives best practices when identifying health conditions, and delivering healthcare services for those conditions. The PHP system further generates or adjusts 106 a PHP (e.g., including episodes of care, health goals, and transitions) for the member based upon the changing member data and the best practice data, and notifies 108 a care team associated with the member (e.g., the care team may be determined by the PHP system) of the health conditions of the member along with the recommended care delivery services for that member. The member may also be notified.

In various embodiments, the PHP system utilizes a plurality of specialized ML and AI models to provide the personalized health plan for the member and/or group. The specially trained ML and AI models include managed care models that are a series of decision support tools that are algorithmically aligned to provide automation for health-oriented objectives. Models of the PHP system are configured to execute a plurality of linear tasks to provide personalized health plans. For example, the models generate outputs, predictions, and recommendations based on the specialization of the model. The models include observation models (e.g., sometimes referred to as oversight models or surveillance models), service models, assurance models, transition models, and/or workflow models. The specialized models enable the PHP system to process individual health plans while also addressing cohort and community healthcare planning. The PHP system utilizes each type of model to provide a single transaction system for digital data transformations using the PHP system.

For example, the PHP system centralizes all member data and facilitates digital data transformations. The digital data transformation provides a flexible, configurable, reliable platform for each personalized health plan and supports the implementation of advanced analytics and content distribution. For example, the PHP system includes digital data transformation that results in a decreased need for prior authorizations of healthcare service or medicines and a reduction in redundancies for further reviews and appeals.

In various embodiments, the PHP system is configured to process member health data to generate a PHP. For example, the PHP system is configured to identify key features from data stored within a database of a member's health history and the current situation of the member with regard to transitions in care. The PHP system processes outputs from adjacent care models and identifies best practices based on past evidence and prior successful management of similar conditions to generate the PHP for the member. The PHP for the member may also be further refined by member inputs into the PHP system. The member inputs include goals and details of the members' health assessments. The PHP system generates recommendations for care, eliminates duplicative data, and stratifies urgency required for healthcare services in each PHP. The PHP system generates notifications for high touch, low touch, and invisible actions for the member based on the processed data. High tough notifications include manual actions to be performed by the member or a member of the care team. A low touch action includes transmitting messages to the member or a care provider. Invisible actions include initiating automated actions by the PHP system.

The PHP system includes tools such as dashboards and checklists to provide advanced analytics and content based on the PHP for each member. The PHP system collects and processes member data using other data such as, for example, population data and person observation data. The member data is then used to identify, prioritize, and format the PHP for the member. For example, the PHP system is configured to provide interventions, programs, and campaigns for members based on the collected data. Additionally, the PHP system provides presumptive care and prevention solutions for members to promote health maintenance and preventative services while also maintaining healthcare duties under conventional methodologies to provide care to the highest need conditions.

Further, the PHP system processes demographics, member health history, and best practices for care to identify and align the PHP to the member's individualized needs. The PHP system is also configured to adjust and modify the PHP based on episodes of care, member goals for their PHP, and their care transitions. In some embodiments, the PHP system processes the data to populate the PHP for the member. The PHP system further notifies the corresponding care teams in response to the changes to the PHP for each member. In some embodiments, the care team populates part of the PHP based on their interactions with the member. As updates are made to the PHP system, the PHP system will translate the data into a common format and push the updates out to the appropriate managers and care providers so they are all informed of the updates in healthcare services being delivered.

In the exemplary embodiment, the PHP system is also configured to generate a user interface for actionable real-time and near real-time dashboards, generate personalized care plans, execute facilitated workflows through embedded algorithms, and automate healthcare programs and campaigns. The healthcare providers are then able to support members using the PHP system by providing input about the member data and matching them with the appropriate care. The input may then be used to further train the AI tools used by the platform for future interactions with the member.

The PHP system utilizes a plurality of managed care models. The managed care models are configured to interact with the members. The managed care models include a series of decision and support tools that are algorithmically aligned to provide automation for member health-oriented objectives. For example, the PHP system includes an oversight or observation model, one or more service models, an assurance model, a transition model, and a workflow model. The observation model is configured to monitor and predict member needs. The one or more service model is configured to automate services for the member such as providing or delivering healthcare services that are recommended for the member. The assurance model is configured to reconcile member data and ensure quality of care and effectiveness of the PHP for the member. The transition model is configured to recognize and facilitate the transition activities affecting the member to ensure seamless care with the PHP. The workflow model supports the care team to provide both conventional healthcare as well as new developments. Each model is described herein in further detail.

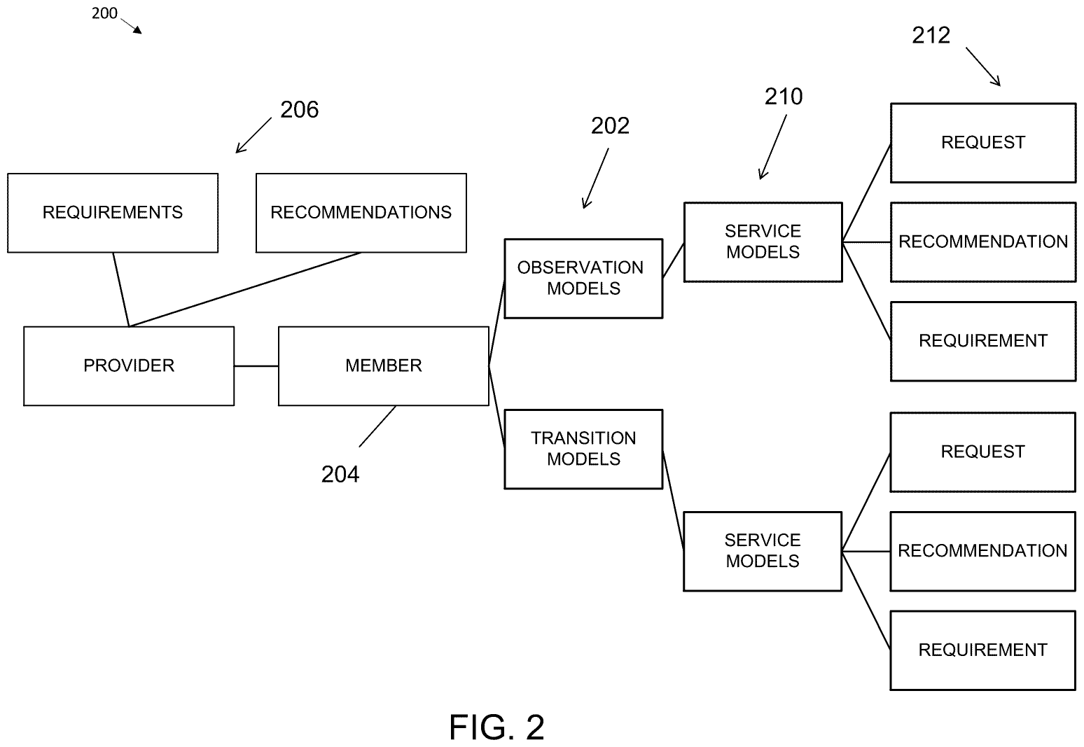

FIG. 2 illustrates an example flow diagram 200 of a coordination between a healthcare provider and a member as implemented by the PHP system. As shown in FIG. 2, observation models 202 gather member data 204 and best practice data 206 (e.g., including requirements and recommendations, such as historical requirements and historical recommendations) associated with a provider 208. Observation models 202 (e.g., including transition models configured to recognize and facilitate the transition activities affecting the member to ensure seamless care with the PHP) transmit gathered data to service models 210 that generate (e.g., and transmit) requests, recommendations, and requirements 212 based upon the data received from observation models 202.

The PHP system is built to provide services to a member of the PHP. For example, the PHP system is connected to a database storing data associated with one or more members. The database includes member information including previous claims, medications, past visits, diagnoses, medical problems, durable medical equipment (DME), lab results, healthcare effectiveness data and information set (HEDIS) care gaps, PHP model outputs, health assessments, and health literacy information. Requests and recommendations are directed to the member from healthcare providers. The PHP system utilizes observation models and transition models to identify and initiate service models. The service models process requests, recommendations, and requirements to provide the PHP to the members. In various embodiments, the observation models and transition models facilitate the interaction between the member and the provider based on the requests, recommendations, and requirements from the service models.

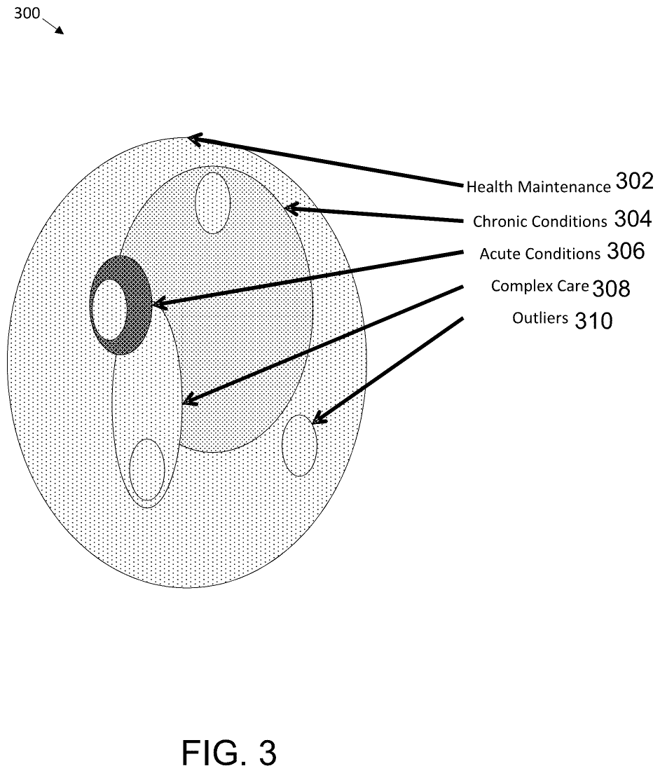

FIG. 3 illustrates an example framework 300 of an observation model (e.g., observation models 202) of the PHP system. As shown in FIG. 3, the observation model is configured to monitor and predict healthcare needs of a member. For example, the observation model is configured to monitor and predict healthcare needs including general healthcare maintenance 302, chronic conditions 304, acute events 306, complex care situations 308, and conditions outside predicable healthcare needs and legacy healthcare practices 310 (e.g., outliers). The observation model is configured to monitor and predict member healthcare needs based on member data including age, gender, family history, and other indicators of health. The observation model generates recommendations for wellness programs, screenings, immunizations, networks, and resources for the PHP based on the member data and best practice data.

The observation model is further configured to monitor and predict healthcare needs based on member chronic conditions. For example, the observation model monitors and predicts healthcare needs related to hypertension (HTN), diabetes mellitus (DM), osteoarthritis (OA), cardiovascular disease (CVD), chronic kidney disease (CKD), behavioral health disorders (BH), substance abuse, and chronic pain. The acute events include exacerbations, outbreaks, and accidents. The outliers and exclusions are monitored within the surveillance models to identify health conditions and issues that are outside of predictable need or representative of legacy healthcare practices such as recidivism, abuse and neglect, unmatched conditions, and unplanned conditions. Further, complex care conditions include multiple conditions, high disability syndromes, transplants, oncology, and behavioral health diseases.

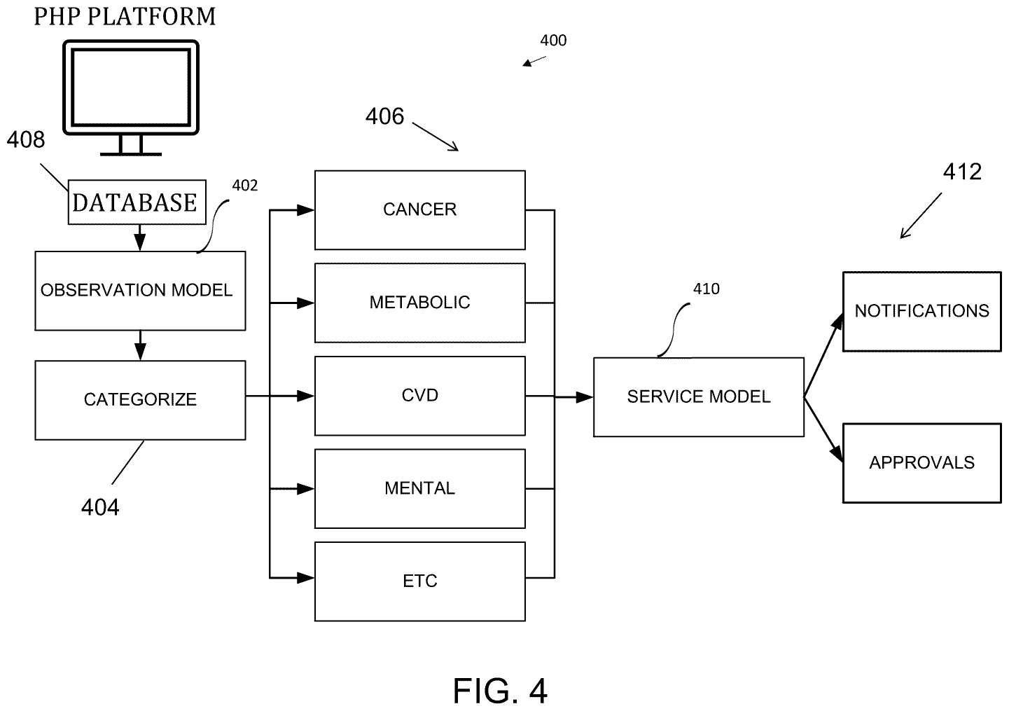

FIG. 4 illustrates an example flow diagram implemented in part by an observation model 402 of the PHP system. In the example embodiment, observation model 402 is used for health maintenance. Observation model 402 is configured to continuously monitor a member's clinical history and evidence library. Observation model 402 processes member data to recognize and generate recommendations for the member. In various embodiments, observation model 402 categorizes 404 the recommendations into screening and wellness categories 406. For example, observation model 402 processes the member data to identify user screening and maintenance needs. The outputs generated by observation model 402 initiate service models 410 that correspond to the identified conditions from observation model 402.

For example, observation model 402 is connected to a database 408. Observation model 402 detects and categorizes 404 a change in a user's health. For example, a change may include a detection of cancer, a change in metabolic health, a change in cardiovascular health, a change in mental health, or other a change in another health-related condition. Observation model 402 processes the detected change to determine if there is a need for a healthcare service. When a need for a healthcare service is identified, observation model 402 identifies a corresponding service model 410 for the identified need to provide the proper notifications and approvals 412.

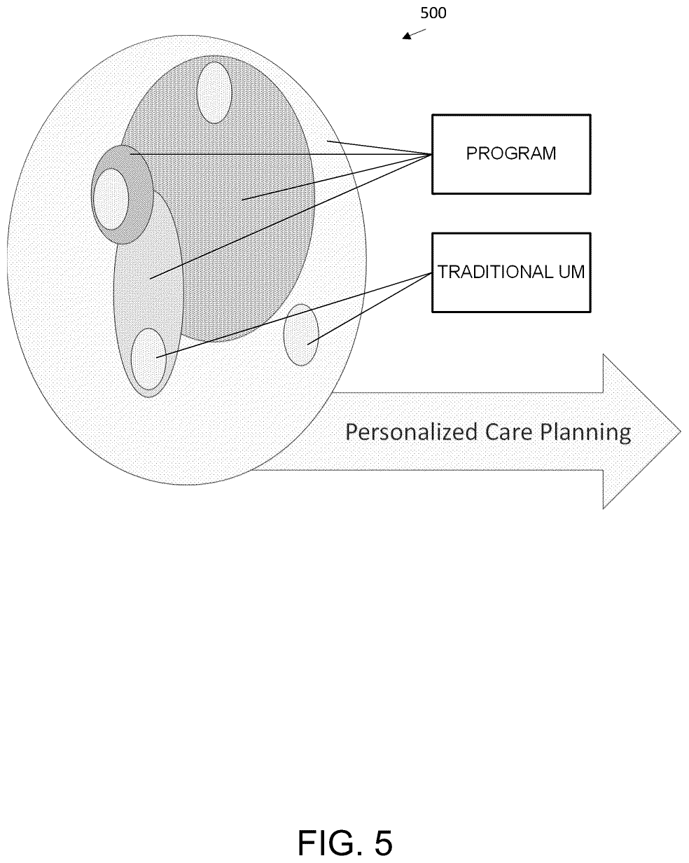

FIG. 5 illustrates an example framework 500 of a service model of the PHP system. As shown in FIG. 5, the service model is configured to automate healthcare services for a member. For example, the service model is configured to provide personalized care planning 502, program enrollment 504, and facilitation of conventional utilization management pathways 506 for providing healthcare services to members. Additionally, the service model is configured to identify opportunities to automatically bundle, package, and deliver healthcare solutions to the member for automatic provisioning of the healthcare services.

In various embodiments, the service model is initiated by an output from a surveillance model. The service model processes the output from the surveillance model to recognize actionable recommendations and provide automated services. In some embodiments, the surveillance model facilitates workflows and generates approvals and notifications for healthcare services. For example, the notifications may include transmitting external messages to members and providers. Additionally, the service model provides notifications internally on a dashboard indicating member needs that have been identified and facilitated. The internal notifications provided to the PHP dashboard incorporate human in the loop (HITL) strategies to provide healthcare service recommendations to the members. In various embodiments, the service model approvals are also recommended from conventional workflow pathways. The provided service may not be linked to the member until performance of the services have been confirmed.

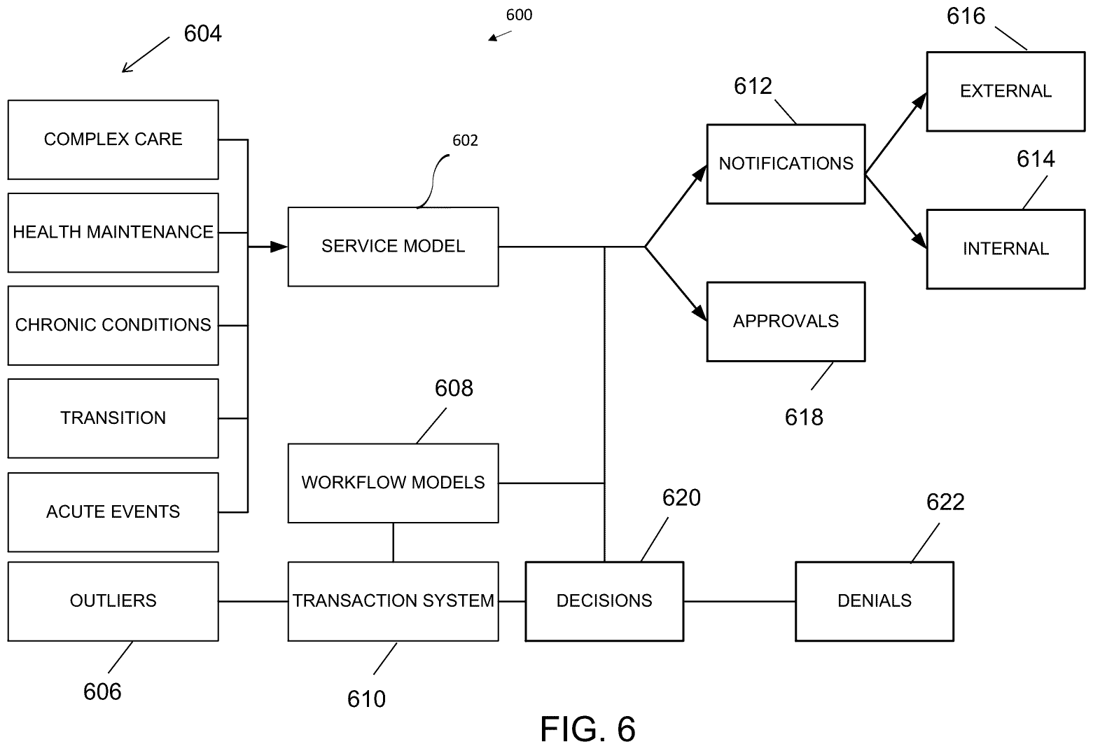

FIG. 6 illustrates an example flow diagram 600 implemented by a service model 602 of the PHP system. For instance, FIG. 6 illustrates a partially more detailed view of flow diagram 400 shown in FIG. 4.

For example, service model 602 is configured to identify a service need 604 from an observation model. The identified service need may include health maintenance, chronic conditions, acute events, complex care cases, and transitional care. Service model 602 also facilitates outlier conditions 606 received from a workflow model 608 on a UM transaction system 610. Service model 602 processes the needs to generate notifications 612 (e.g., internal notifications 614 and external notifications 616) and approvals 618. In various embodiments, service model 602 is configured to receive decisions 620 to facilitate denials 622 for service.

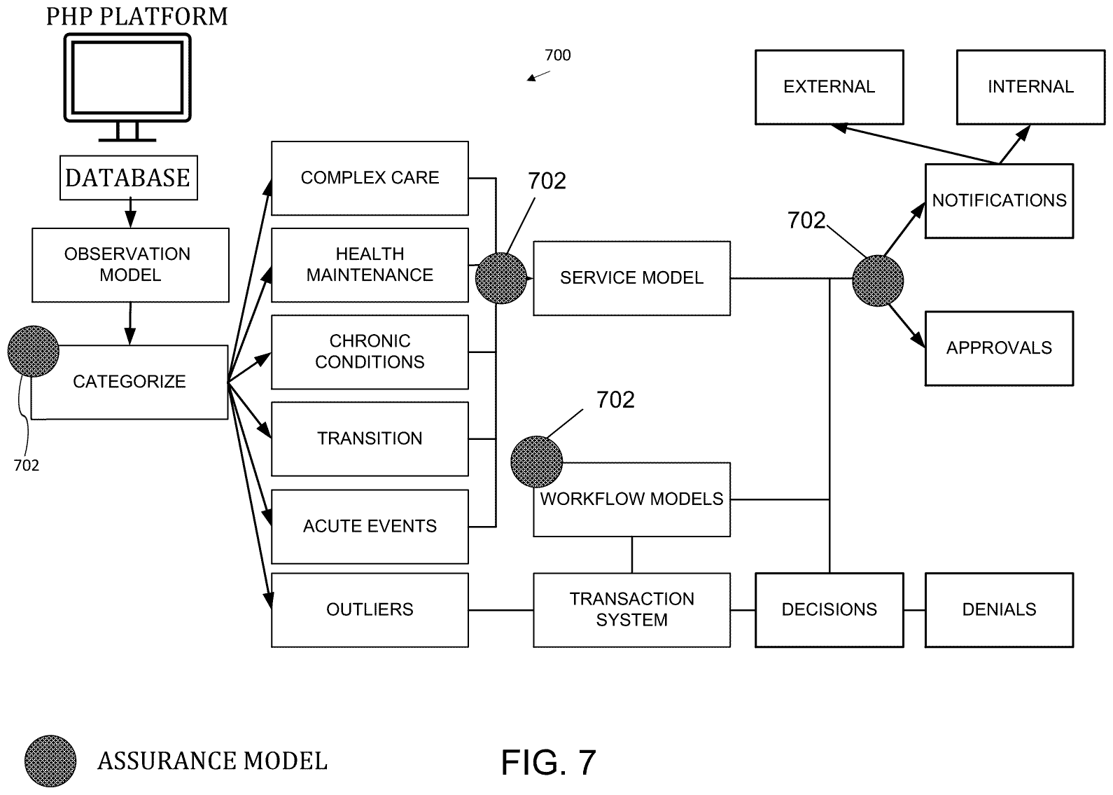

FIG. 7 illustrates an example flow diagram 700 including an assurance model 702 of the PHP system. For example, flow diagram 700 illustrates a partially more detailed view of flow diagrams 400, 600, shown in FIGS. 4 and 6, and illustrating various example implementations of assurance model 702.

As shown in FIG. 7, assurance model 702 is configured to verify healthcare recommendations to ensure quality of care, reduce bias in healthcare services provided, and prevent redundancies in the provided healthcare services. For example, assurance model 702 is configured to de-duplicate and reconcile member data stored in a database. Assurance model 702 is configured to target outcomes for a member to identify gaps, trends, and performance of the healthcare services provided to the member. For example, assurance model 702 verifies quality standards of the provided healthcare services, and aggregates cohorts. Further, assurance model 702 is implemented where multiple recommendations intersect. For example, assurance model 702 is deployed where a member receives multiple recommendations from doctors to align the PHP. Assurance model 702 is further configured to confirm quality measures in recommendation by the PHP models.

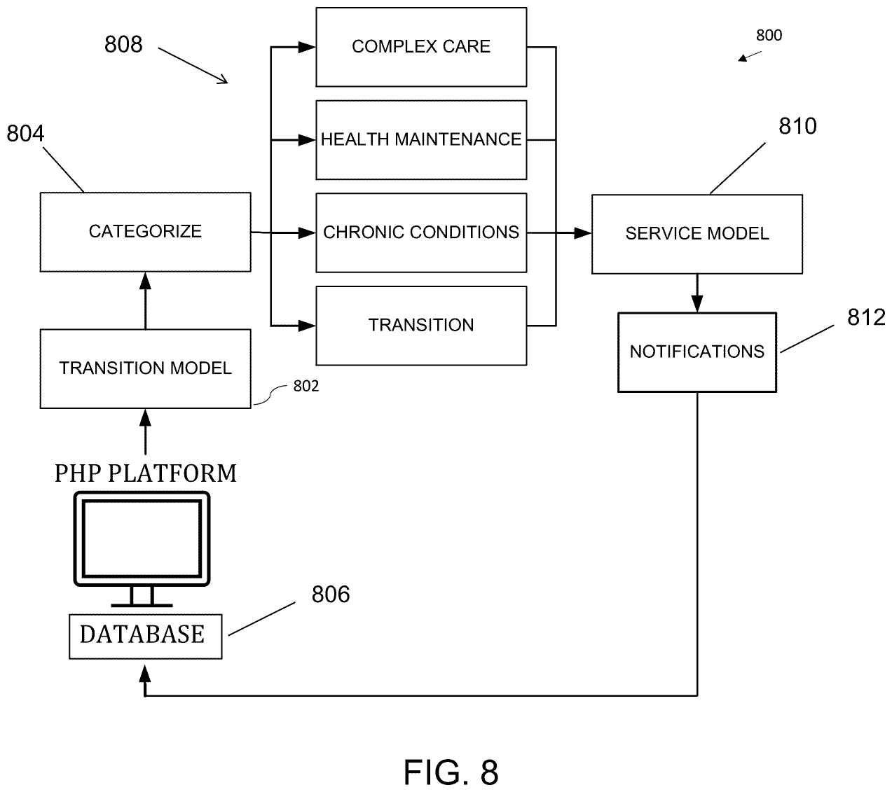

FIG. 8 illustrates an example flow diagram 800 including a transition model 802 of the PHP system. For example, FIG. 8 illustrates a transition model flow similar to part of flow diagram 200.

As shown in FIG. 8, transition model categorizes 804 a transition in member health based upon member health data (e.g., based upon data received from a database 806 or other device) into various example categories 808. After categorization, the categorized member health data is transmitted to a service model 810 configured to generate and transmit notifications 812 based upon the transition in member health. Outputs from service model 810 may be stored in database 806.

Transition model 802 processes member data corresponding to member care to provide situational awareness to the member about the provided healthcare services. For example, transition model 802 is configured to monitor adjuvant surveillance or ongoing surveillance following a medical procedure. Further, transition model 802 is configured to recognize changes in member conditions, venues, and roles of the provided healthcare services. Transition model 802 generates and applies adjustments to member profiles and care pathways to queue anticipated healthcare service needs and workflows. Further, transition model 802 verifies quality standards for care, aggregates cohorts, aligns healthcare outcome goals, monitors program adherence, and recommends campaigns and engagement.

FIG. 9 illustrates an exemplary embodiment of a dashboard 900 (e.g., a graphical user interface (GUI)) that may be generated by the PHP system and displayed on a display device associated with PHP system.

As shown in FIG. 9, the models of the PHP system provide information to dashboard 900 to provide HITL capabilities to the PHP system. The aggregated information from the models enables providers to monitor communities of members for health indicators and interventions using near-real time dashboards 900.

For example, the aggregated information is based on cohorts, demographics, programs, and campaigns associated with the members to monitor the communities. Dashboard 900 is configured to display notifications to the care team to provide care management services. For example, the notifications are generated from the service model to provide information to the care team associated with the provided healthcare services. Dashboard 900 includes outputs from the PHP models to facilitate healthcare processes. In various embodiments, the dashboard 900 also includes utilization management processes to facilitate outliers and exclusions to ensure all data is centralized to the dashboard.

In various embodiment, dashboard 900 facilitates prior authorization information 902. The prior authorization information includes indications for how many prior authorizations are due, how many prior authorizations are new, and how many prior authorizations are for review. Dashboard 900 also facilitates concurrent review indications 904 such as reviews older than a certain time interval, new concurrent reviews, and peer-to-peer (p2p) reviews. Dashboard 900 further indicates appeals 906 that are new and due. Dashboard 900 is also configured to provide an indication corresponding to audits 908 from Medicaid, Medicare, and medical policy audits.

In various embodiments, dashboard 900 facilitates PHP care management. Dashboard 900 includes information about inpatient (IP) information 910 including acute care (AC) data, skilled nursing facility (SNF) data, long-term acute care (LTAC) data, and rounds or review data. Dashboard 900 also includes venue transfer information (Venue Trx) 912 including transfer review data, planned transfer data, and a transfer progress data. Dashboard 900 also includes careplan information 914. The careplan information includes the review data, member location, and quality score data. Dashboard 900 further includes social transaction data 916 including transactions for review, number of members being cared for, and an indication of a scheduled care action such as rounds. Dashboard 900 also includes provider data 918 including review data, members data, and an indication of rounds to be performed. In various embodiments, the dashboard includes engagement data. The engagement data includes program data 920, campaign data 922, and a cohort data 924. Dashboard 900 provides facilitated workflows for the information displayed on dashboard 900 through embedded algorithms. In some embodiments, dashboard 900 provides automated programs and campaigns through the dashboard.

For example, dashboard 900 facilitates prior authorization processes, concurrent review processes, and appeal processes for members. Dashboard 900 is also configured to facilitate additional claim management processes based on the outputs from the models. Dashboard 900 also facilitates processes for rounds, transitions, care planning, member outreach, and care navigation. In various embodiments, dashboard 900 facilitates population health processes. For example, dashboard 900 facilitates cohorts, provides program management, and executes campaigns. Dashboard 900 is further configured to provide technology support to test and audit the technology associated with the PHP system.

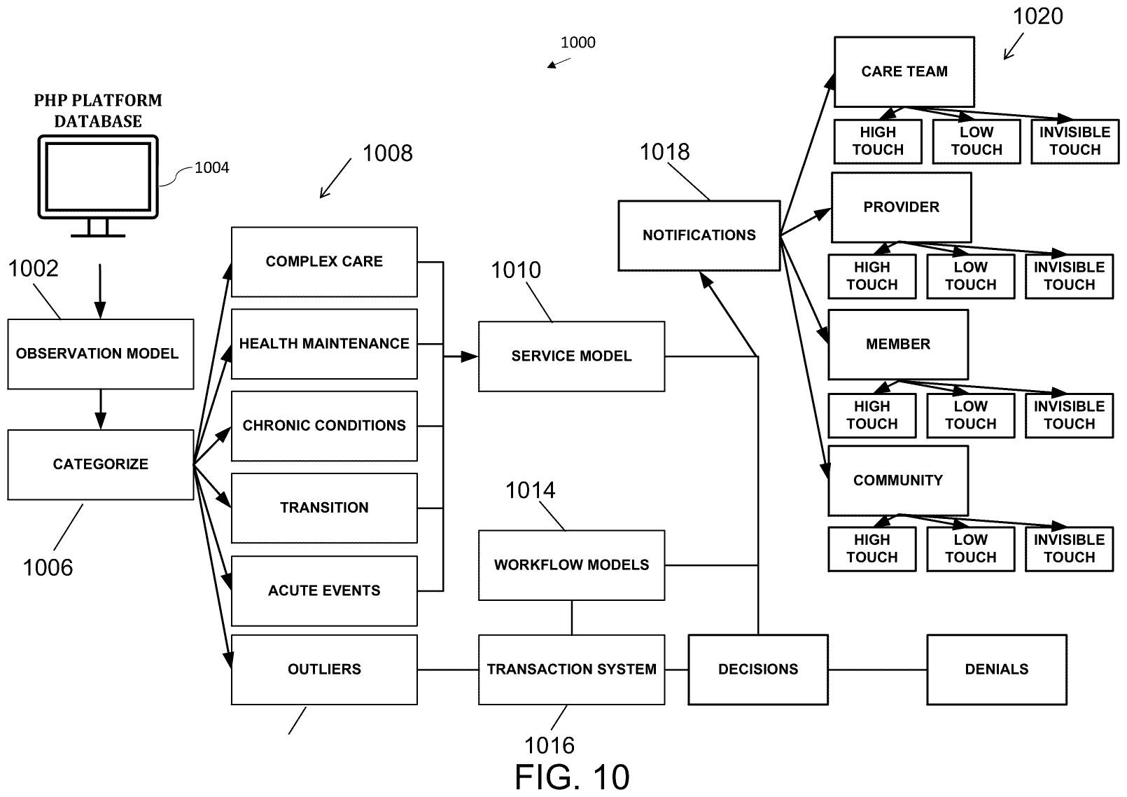

FIG. 10 illustrates an example flow diagram 1000 of an example embodiment of a notification process implemented by the PHP system, in accordance with the present disclosure. For instance, flow diagram 1000 includes a partially more detailed view of flow diagram 700.

In various embodiments, the observation models 1002 are connected to a database 1004 for storing data associated with one or more members. Observation models 1002 process the member data to detect changes in the member data. When a change is detected by observation models 1002, observation models 1002 (e.g., or another computer component of the PHP system) categorizes 1006 the detected change. For example, the categorized change categories 1008 include health maintenance, chronic conditions, complex care, and transitions.

The PHP system processes the changes detected by observation models 1002 to determine if there is a member need associated with the change. When there is a need associated with the change, a service model 1010 determines what next action (e.g., a next best action based upon historical data) to take based upon the change. Next best actions include transmitting notifications to the members, processing approvals for care, and determining when human decisions are required to provide services to the member.

In some embodiments, when the detected change by observation model 1002 is an outlier 1012, the PHP system utilizes an improved UM system. The PHP system improves upon a conventional UM process by leveraging workflow models 1014 to facilitate transactions systems 1016.

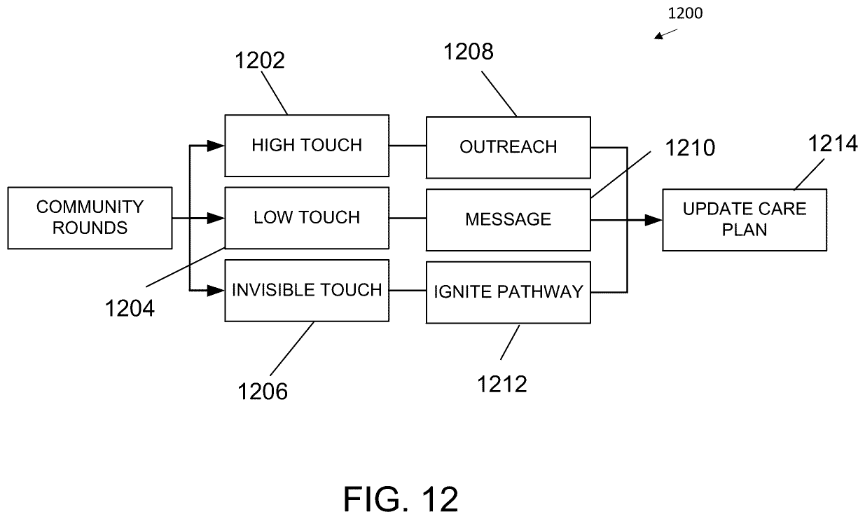

Service models 1010 are configured to sort notifications 1018 based on where a notification 1018 is being distributed. For example, service models 1010 may determine distribution of notifications 1018 should be provided to various groups 1020 such as care team, providers, members, and communities. Further, service models 1010 determine type of notification to distribute. For example, the PHP system (e.g., service models 1010 thereof) may determine to distribute high touch, low touch, or invisible touch notifications, as shown in FIG. 10 and as explained herein (e.g., see FIGS. 12-14).

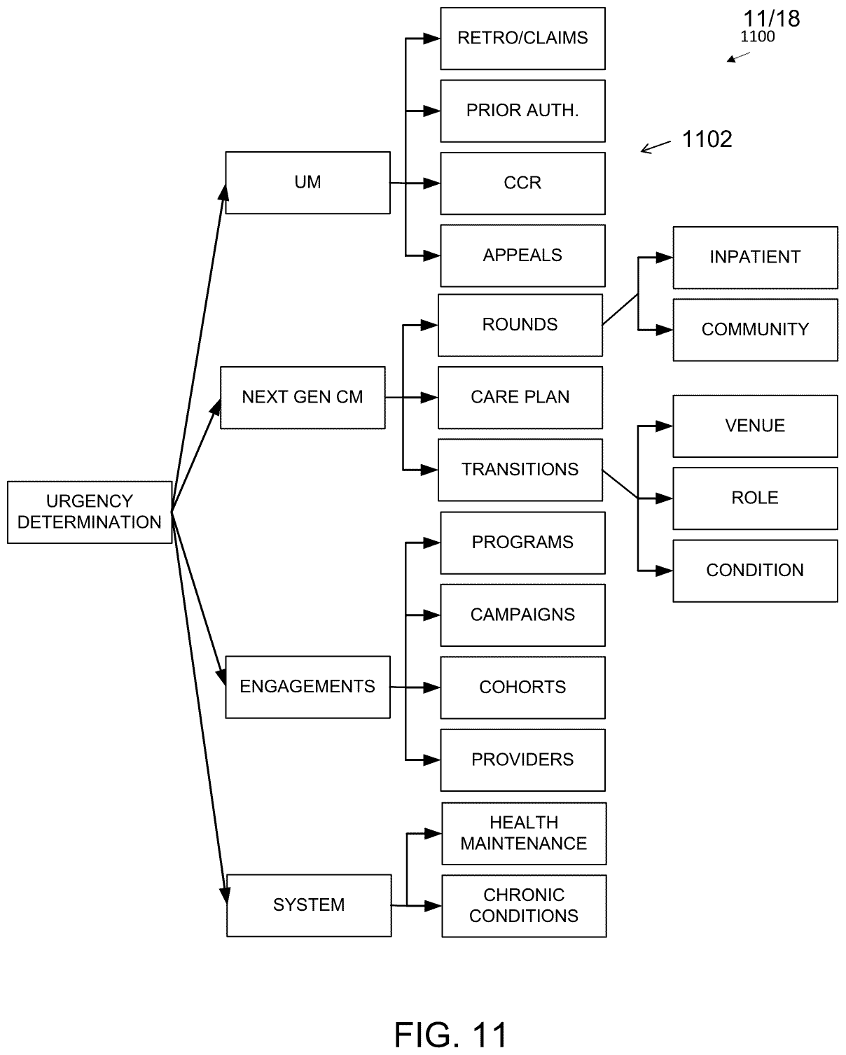

FIG. 11 illustrates an example embodiment of a flow diagram 1100 for urgency determination of the PHP system (e.g., by service models), in accordance with the present disclosure.

For example, service models may determine the urgency of a tasks determined by the service models based upon inputs from observation models (e.g., and displayed on a dashboard). Tasks 1102 may include UM tasks such as retrospective claim review, prior authorizations, concurrent reviews, and appeals. The tasks may also include system audits and tests. The tasks may also include and facilitate claim management tasks such as rounds, transitions, and care plans. The rounds include inpatient rounds and community rounds. The tasks may be enrollment in engagement information such as educational programs, campaigns, cohorts, and providers.

Other example tasks may include providing medical devices to members in order for member health data to be monitored in real time by the PHP system. As an example, service models may determine to provide wearable devices to certain members that are configured to communicate with the PHP system. Accordingly, a member is wearing the device, observation models can track the member's health data in real time and pass the health data through the various processes described herein in real time and update member care (e.g., the PHP associated with the member) in real time.

In the example embodiment, different tasks 1102 are associated with different levels of urgency determined by service models based upon inputs from observation models. Based upon the urgency determined, different actions are taken by the PHP system (e.g., high touch, low touch, or invisible touch, as explained herein).

FIG. 12 illustrates an example flow diagram 1200 for an example embodiment of distributing notifications for community rounds by the PHP system. For instance, flow diagram 1200 illustrates notifications for community rounds resulting from the process shown in flow diagram 1100 of FIG. 11 ending at “COMMUNITY” from “ROUNDS”.

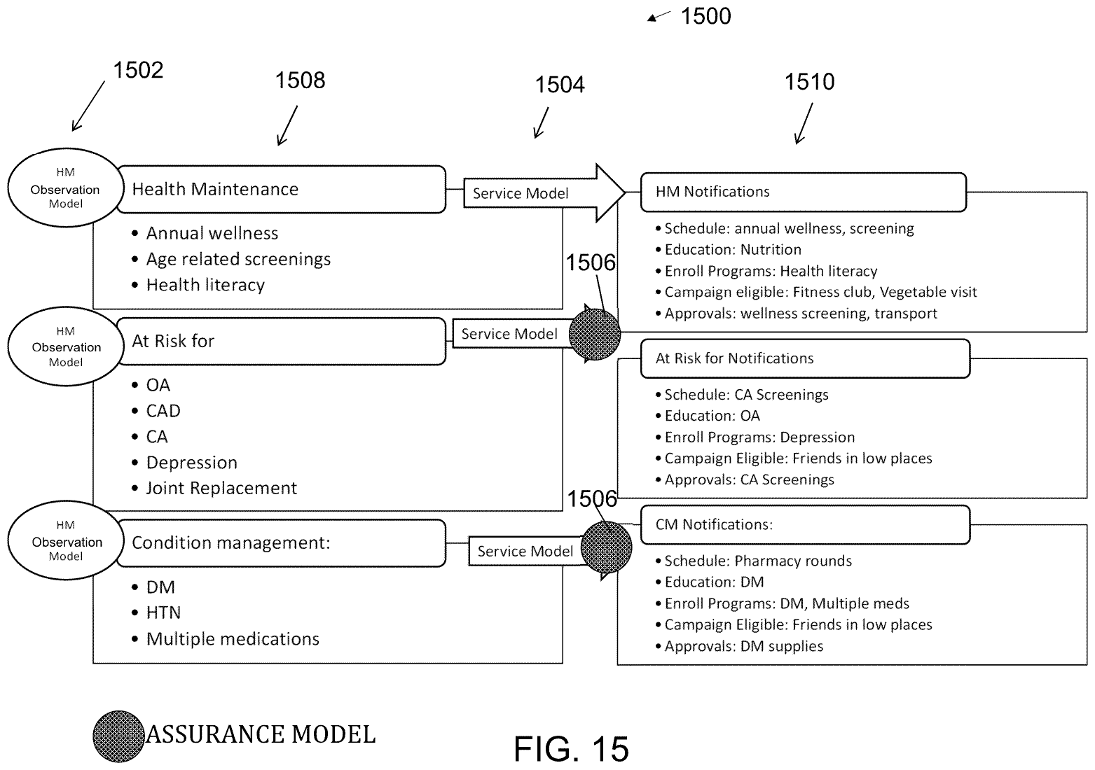

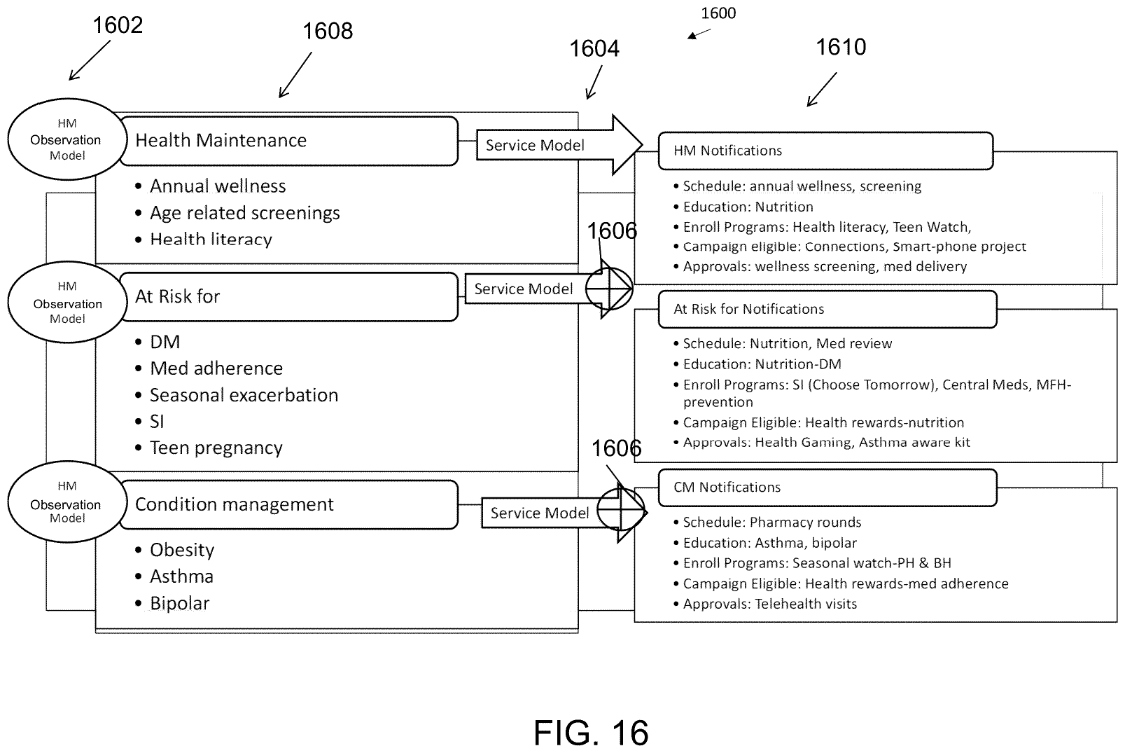

A service model determines, based upon the determined level of urgency explained herein, a level of notification including high touch 1202, low touch 1204, and invisible touch 1206 community outreach programs and updates the care plan accordingly. High touch 1202 outreach includes enabling outreach activities 1208 to the community (e.g., scheduling appointments, procedures, transportation to/from by communicating with third party transportation services, scheduling education, automatically approving certain procedures, see FIGS. 15 and 16, and notifying a member thereof). Low touch 1204 outreach includes creating and transmitting messages 1210 to the community (e.g., without automatically scheduling follow ups). Invisible community outreach includes igniting pathways 1212 within the community (e.g., not sending a notification (e.g., no action taken) because of a low level of urgency). Once the determined task has been performed, the care plan is updated 1214.

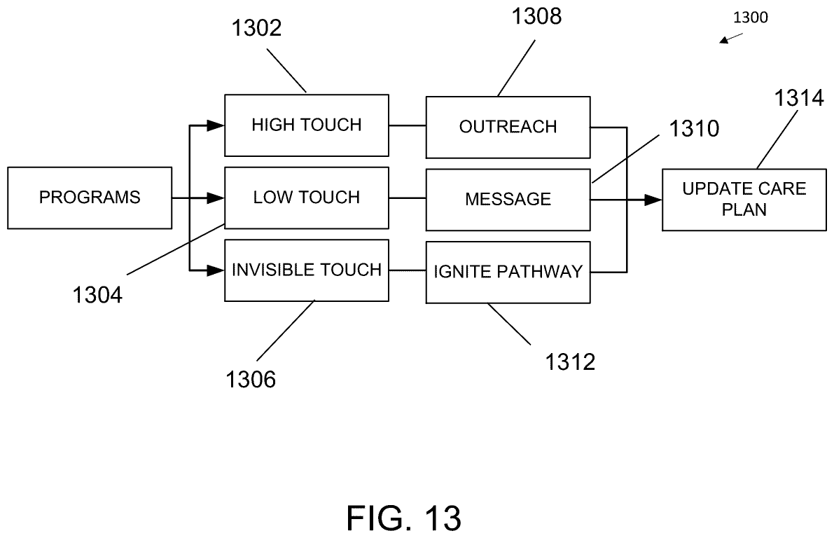

In another example, FIG. 13 illustrates an example flow diagram 1300 for an embodiment of distributing notifications for member programs by the PHP system. For instance, FIG. 13 illustrates notifications for programs resulting from the process shown in flow diagram 1100 of FIG. 11 ending at “PROGAMS”.

In some embodiments, programs include population health programs. The population health programs are associated with condition and venue focused objectives. The engagement programs implement operations based on centers of excellence including case reviews by medical directors. The case reviews include reviews of members and providers performance based on the program objectives.

A service model determines, based upon the determined level of urgency explained herein, a level of notification including high touch 1302, low touch 1304, and invisible touch 1306 community outreach programs and updates the care plan accordingly. High touch 1302 outreach includes enabling outreach activities 1308 (e.g., automatic enrollment in programs, see FIGS. 15 and 16, and notifying a member of the enrollment). Low touch 1304 outreach includes creating and transmitting messages 1310 (e.g., notifying a member of programs). Invisible community outreach includes igniting pathways 1312 (e.g., no further actions taken by the PHP system) because of a low level of urgency). Once the determined task has been performed, the care plan is updated 1314.

The types of outreach include high touch outreach, low touch messaging outreach, and invisible outreach including igniting pathways to facilitate the outreach. When the outreach is completed, the corresponding care plan is updated.

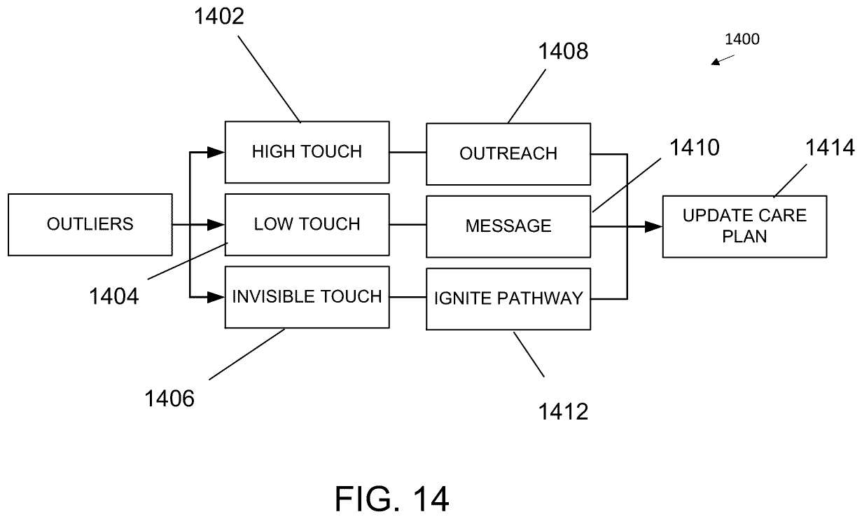

FIG. 14 illustrates an example flow diagram 1400 of distributing notifications for outliers of a cohort. For instance, flow diagram 1400 illustrates notifications for outliers resulting from the process shown in flow diagram 1100 of FIG. 11 ending at “COHORT”.

Care teams share responsibilities for oversight of community cohorts. Community engagement includes reviewing and managing cohort (e.g., group of members associated with the community engagement) performance to drive community focused outcomes. The PHP system reviews key performance indicators (KPIs) for community engagement and identifies outliers. The PHP system (e.g., a service model) then determines whether to engage the cohort through high touch 1402, low touch 1404, or invisible engagement 1406. For instance, high touch 1402 may include automatic outreach 1408, such as automatically enrolling members of the community in programs associated with the community focused outcome and notifying the members of the enrollment (e.g., see FIGS. 15 and 16), low touch may include messaging 1410 members of the community about programs associated with the community focused outcome, and invisible touch may include “igniting the pathway” 1412 for the community focused outcome (e.g., no action taken) in PHPs for members of the community because of low urgency. Once the determined task has been performed, the care plan is updated 1414.

FIG. 15 illustrates an exemplary flow diagram 1500 of a personalized health plan using the PHP system. For instance, FIG. 15 illustrates an example PHP for a member as determined via observation models 1502 and service models 1504 (e.g., and assurance models 1506), as explained herein.

The PHP system processes member data to generate the PHP based on the member's health data. For example, the PHP system processes medical data for a member that is a 52 year old female. The member's past medical history (PMH) includes type 2 diabetes, high blood pressure, psoriatic arthritis, and obesity. The member's past surgical history (PSH) includes gallbladder removal and two back surgeries. The member has a family history (FH) of heart disease, colon cancer, breast cancer, obesity, and substance abuse. The social history of the member (SH) is an unmarried, non-smoker who works remotely and lives in a suburban area. The member's medications include six oral medications and one injection. The member uses durable medical equipment. In the last twelve months, the member has had no hospitalization, one emergency room visit, one change in medication, no provider visits, and 12 physical therapy visits. The member faces racial and ethnic risk for cardiovascular disease and dementia. The member relies on themselves for transportation opportunities and has low health literacy.

Upon processing the member data, the PHP system generates an individualized PHP for the member and provides appropriate notifications. For example, observation models 1502 group the data based on health maintenance data, health risk data, and health condition management data. An observation model provides outputs 1508 including recommendations for annual wellness visits, age related screenings, and suggestions to improve health literacy. A service model 1504 is identified based on outputs 1508 from observation model 1502. For example, service model 1504 processes the health maintenance data to execute actions 1510 based on the health maintenance notifications. The health maintenance notifications are utilized by the service module to schedule annual wellness visits and screenings based on the outputs 1508.

The PHP system also identifies the health risk member data using a observation model 1502. For example, observation model 1502 provides outputs 1508 identifying that the member is at risk for osteoarthritis, coronary artery disease, cancer, depression, and joint replacements. Observation model 1502 (e.g., or another component of the PHP system) utilizes outputs 1508 from observation model 1502 to identify a service model 1504 to assist with the health risks. In some embodiments, service model 1504 includes an assurance model 1506 to verify the recommendations of observation model 1502 and verify the actions of the service model 1504 (e.g., to prevent duplicate actions, scheduled appointments, notifications).

Service model 1504 determines actions 1510 based upon outputs 1508 from observation model 1502 including scheduling and obtaining approval for cancer screenings, facilitating osteoarthritis education, enrolling the member in programs for depression, and enrolling the member into campaigns.

Observation model 1502 also processes condition management data. For example, observation model 1502 identifies the diabetes, hypertension, and multiple medication data for a condition management service model 1504. Assurance model 1506 verifies outputs 1508 from observation model 1502 and prevents duplicate actions being taken by service model 1504 (e.g., weight loss education may be determined by service model 1504 as separate actions to be taken for both diabetes and hypertension, but assurance model 1506 would deduplicate the duplicative weight loss educations and cause service model 1504 to only provide one notification for weight loss education instead of multiple).

Service model 1504 determines actions 1510 based upon outputs 1508 from observation model 1502 including scheduling pharmacy rounds, providing diabetes education, identifying and enrolling the member in campaigns, and seeking approvals for medical supplies such as diabetes supplies. In this way, observation models 1502, service models 1504, and assurance models 1506 provide a PHP to a member based on their specific healthcare needs that the PHP system extracts from the members medical health data.

FIG. 16 illustrates an additional exemplary flow diagram 1600 of a personalized health plan implemented using the PHP system. In this example, the member has a medical history of asthma, obesity, ADHD, and is bipolar. The member does not have paroxysmal sympathetic hyperactivity (PSH) and unknown family history (FH). The member has a history of missing and changing primary care provider and pharmacy interactions due to location changes across the state. For example, the member moves between counties throughout the state based on their residential situation and their placement through the department of social services (DSS). The state is the guardian of the member, however, the member's family remains involved and retains some rights. The member is prescribed 9 medications, with three of them taken as needed. The prescriptions include psychotropics. The twelve-month utilization of the member includes three residential setting changes including therapeutic foster care (TFC) and psychiatric residential treatment facilities (PRTF). The member has racial and ethnic risk for diabetes, hypertension, cardiovascular disease, and obesity. The member is dependent on others for their transportation and their health literacy is immeasurable.

Observation models 1602 analyze the member data (e.g., described above), and the PHP system provides an individualized PHP for the member. For example, observation models 1602 group/categorize the member data based on health maintenance data, health risk data, and health condition management data. Observation model 1602 provides outputs 1608 including recommendations for annual wellness visits, age related screenings, and suggestions to improve health literacy. A service model 1604 is identified based on the outputs of observation model 1602.

For example, service model 1604 processes the health maintenance notification to execute actions 1610 based on the health maintenance notifications. As an example, service model 1604 to schedules annual wellness visits and screenings based on the health surveillance model output. Service model 1604 is further configured to provide educational services, enroll the member into programs, identify eligibility for campaigns, and obtain approvals for health maintenance services such as wellness screening and medicine delivery.

Observation model 1602 also identifies the health risk member data. For example, the observation model 1602 identifies in outputs 1608 that the member is at risk for diabetes, medical adherence, seasonal exacerbation, suicidal ideation, and teen pregnancy. Observation model 1602 may utilize outputs 1608 to identify a corresponding service model 1604 to assist with the health risks. In some embodiments, service model 1604 includes an assurance model 1606 to verify the categorizations of observation model 1602 and verify the actions of service model 1604.

Service model 1604 determines actions 1610 to be taken based upon outputs 1608 from observation model 1602. For example, service model 1604 schedules nutrition and medicine review, provides diabetes management nutrition education, enrolls in programs corresponding to the at-risk data, identifies campaigns for the member, and seeks approvals for the member.

The PHP system also analyzes condition management data. For example, observation model 1602 identifies outputs 1608 such as obesity, asthma, and bipolar data and passes outputs 1608 to a condition management service model 1604. Assurance model 1606 may monitor condition management activities performed by observation model 1602 and service model 1604, as explained herein. Service model 1604 associated with condition management, and overseen by assurance model 1606, is configured to perform actions 1610 such as scheduling pharmacy rounds, providing asthma and bipolar education, identifying and enrolling the member in campaigns, and seeking approvals for telehealth visits. In this way, observation models 1602, service models 1604, and assurance models 1606 provide a PHP to the member based on their specific healthcare needs that the PHP system extracts from the members medical health data.

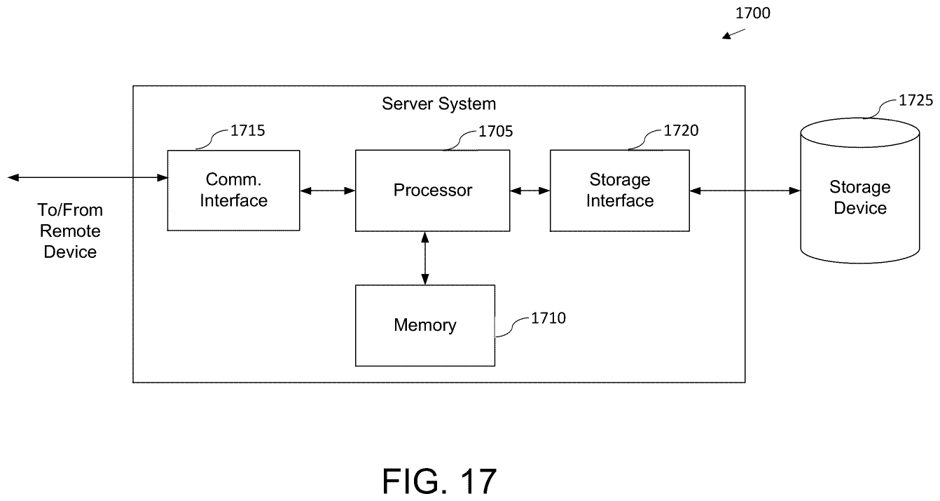

FIG. 17 illustrates an example configuration of a server system 1700. Server system 1700 may be used to implement the PHP system, for example. Server system 1700 includes at least one processor 1705 for executing instructions. Instructions may be stored in a memory area 1710, for example. Processor 1705 may include one or more processing units (e.g., in a multi-core configuration) for executing instructions. The instructions may be executed within a variety of different operating systems on server system, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

Processor 1705 is operatively coupled to a communication interface 1715 such that server system 1700 is capable of communicating with a remote device or another server system. For example, communication interface 1715 may receive requests from, and send results to a device via the Internet.

Processor 1705 may also be operatively coupled to a storage device 1720. For example, storage device 1720 may be used to implement database 1725. Storage device 1720 is any computer-operated hardware suitable for storing and/or retrieving data. In some embodiments, storage device 1720 is integrated in server system 1700. For example, server system 1700 may include one or more hard disk drives as storage device. In other embodiments, storage device 1720 is external to server system 1700 and may be accessed by a plurality of server systems. For example, storage device 1720 may include multiple storage units such as hard disks or solid state disks in a redundant array of inexpensive disks (RAID) configuration. Storage device 525 may include a storage area network (SAN) and/or a network attached storage (NAS) system.

In some embodiments, processor 1705 is operatively coupled to storage device 1720 via a storage interface. Storage interface is any component capable of providing processor with access to storage device. Storage interface may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor with access to storage device 1720.

Memory area 1710 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

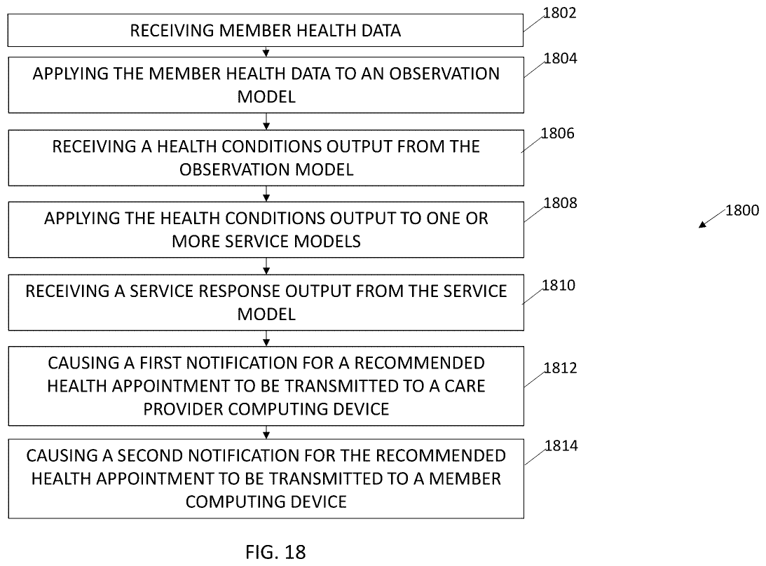

FIG. 18 illustrates an example method 1800 for personalized health planning, in accordance with the present disclosure.

As shown in FIG. 18, method 1800 includes receiving 1802 member health data associated with a member from at least one of a database or a member computing device and applying 1804 the member health data to an observation model, wherein the observation model is trained to identify health conditions of the member based upon historical health data. Method 1800 also includes receiving 1806 a health conditions output from the observation model, the health conditions output including one or more health conditions associated with the member based upon the member health data, and applying 1808 the health conditions output to one or more service models, wherein the one or more service models are trained to generate treatment plans associated with care for different health conditions. Method 1800 further includes receiving 1810 a service response output from the service model, the service response output including a recommended treatment plan including a recommended health appointment for the member based upon a health condition of the one or more health conditions, causing 1812 a first notification for the recommended health appointment to be transmitted to a care provider computing device associated with a care provider to conduct the recommended health appointment for the member, and causing 1814 a second notification for the recommended health appointment to be transmitted to the member computing device to notify the member of the recommended health appointment with the care provider.

In some embodiments of method 1800, the one or more service models are further trained to prioritize health conditions based upon historical severities of the health conditions. In some embodiments of method 1800, the one or more health conditions include a plurality of health conditions, the service response output further includes severities of the plurality of health conditions, and method 1800 further includes causing the first notification and the second notification to be transmitted before other notifications associated with the plurality of health conditions based upon the health condition being a highest priority health condition and causing a third notification to be transmitted to the member computing device after causing the first notification and the second notification to be transmitted, the third notification associated with a next highest priority health condition of the plurality of health conditions and including a recommended treatment to address the next highest priority health condition.

In some embodiments, method 1800 includes receiving updated member health data associated with the member from at least one of the database or the member computing device, the updated member health data including at least one update to the member health data, inputting the updated member health data to the observation model, receiving an updated health conditions output from the observation model, inputting the updated health conditions output to the one or more service models, and receiving an updated service response output from the one or more service models, the updated service response output including an updated recommended treatment plan including a plurality of treatment actions, each treatment action being assigned a priority level.

In some embodiments, method 1800 includes generating a graphical user interface (GUI), the GUI including a first selector indicating that the recommended health appointment has been recommended to the care provider based upon the first notification and one or more second selectors associated with one or more additional appointments assigned to the care provider wherein the GUI is updated in real time based upon outputs associated with the care provider from the one or more service models.

In some embodiments of method 1800, the health conditions output includes duplicates of at least one health condition that were identified based upon different factors in the member health data wherein method 1800 further includes applying the member health data to an assurance model, the assurance model trained to de-duplicate health data based upon historical duplicates identified in the historical health data, and receiving an assurance output from the assurance model, the assurance output including an updated health conditions output including no duplicates of the at least one health condition wherein the health conditions output is updated with the updated health conditions output before the at least one processor applies the health conditions output to the one or more service models.

In the system and method described herein a computer processor or a processing element may be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, statistics and information, and/or historical data. The machine learning programs may utilize deep learning algorithms that may be primarily focused on pattern recognition, and may be trained after processing multiple examples. The machine learning programs may include Bayesian program learning (BPL), voice recognition and synthesis, image or object recognition, optical character recognition, and/or natural language processing—either individually or in combination. The machine learning programs may also include natural language processing, semantic analysis, automatic reasoning, and/or other types of machine learning or artificial intelligence.

In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs.

As described above, the systems and methods described herein may use machine learning, for example, for pattern recognition. That is, machine learning algorithms may be used by the PHP system to automatically classify data and generate messages. Accordingly, the systems and methods described herein may use machine learning algorithms for both pattern recognition and predictive modeling. As an example, the PHP computing system may utilize machine learning techniques when automatically classifying data and generating messages, as described herein.

A processor or a processing element may be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, statistics and information, and/or historical data. For example, the models described herein may work together to assign a “next best” action to a member (e.g., a worker most efficient at that type of work, with availability and qualifications to do that work) based upon the member's health data and severities of the member's health conditions. The model may be refined and improved over time by looping feedback regarding next best actions (e.g., and any identified accuracies and/or inaccuracies in the model output) back in to train the model.

The machine learning programs may utilize deep learning algorithms that may be primarily focused on pattern recognition, and may be trained after processing multiple examples. The machine learning programs may include Bayesian program learning (BPL), voice recognition and synthesis, image or object recognition, optical character recognition, and/or natural language processing-either individually or in combination. The machine learning programs may also include natural language processing, semantic analysis, automatic reasoning, and/or other types of machine learning or artificial intelligence.

In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs.

As described above, the systems and methods described herein may use machine learning, for example, for pattern recognition. That is, machine learning algorithms may be used by the PHP computing system to generate personalized health plans for members. The machine learning algorithms may also be used by the PHP computing system to attempt to predict future needs and to generate and/or store trainings associated with the predicted future needs in order to potentially avoid the predicted future needs (e.g., educational modules and/or videos regarding preventing certain health conditions). Accordingly, the systems and methods described herein may use machine learning algorithms for both pattern recognition and predictive modeling.

In some embodiments, the models described herein may be trained in multiple training stages using multiple training sets. For instance, observation models may be trained with different training sets associated with detecting different conditions. As an example, a first round of training may be performed with historical health training data indicative of heart disease. A second round of training may be performed with historical health training data indicative of diabetes, and so forth. Feedback may be provided to observation models in real time (e.g., regarding correct and/or incorrect categorization of member health conditions based upon member health data) in order for the observation models to be improved in real time.

Service models may also be trained using multiple training sets in multiple stages. For instance, service models may be trained with different training sets associated with assigning severities of different health conditions. As an example, a first round of training may be performed using historical health training data associated with generally healthy members (e.g., where certain relatively minor health conditions developing may be marked as more severe because the members are otherwise generally healthy). A second round of training may be performed using historical health training data associated with generally unhealthy members. For instance, if a member has cancer, a minor change in certain health conditions may not be severe. In some aspects, minor changes for generally unhealthy members may be more severe because the generally unhealthy members may be more prone to minor changes causing severe damage.

Different service models may be generated based upon how they were trained. For instance, a first service model may be trained based upon data for generally healthy members. Accordingly, when an output from an observation model indicates that a patient is generally healthy, that first service model may be applied to that member's health data in order to determine which actions to take. As another example, a second service model may be trained based upon data for members with a certain type of disease. When an output from an observation model indicates that the member has that type of disease, the second service model may be applied to that member's health data in order to determine which actions to take (e.g., certain conditions may be more or less sever and require more or less urgent actions based upon the disease than would be the case for a generally healthy member). Feedback may be provided to service models in real time (e.g., regarding correct and/or incorrect prioritization of actions based upon health outcomes resulting therefrom) in order for the service models to be improved in real time.

As another example, assurance models may also be trained in multiple stages. For instance, an assurance model may be trained in a first stage using historical observation data indicating which health conditions may be duplicative (e.g., so that the assurance model can monitor outputs from observation models in order to de-duplicate data). An assurance model may also be trained in a second stage using historical service data indicating actions (e.g., outputs from a service model) that may be duplicative (e.g., in order to de-duplicate actions before they are taken multiple times). Feedback may be provided to assurance models in real time (e.g., regarding duplicates that still made it through the assurance models and/or de-duplication of data by the assurance models that should not have been de-duplicated) in order for the assurance models to be improved in real time.

Accordingly, the various specific models described herein may each may trained in different stages with different training data in order to provide an efficient PHP system with the various models working together to trigger next best actions for members and to reliably improve the models over time based upon real time feedback.

In some embodiments, a computer program is provided, and the program is embodied on a computer-readable medium. In an example embodiment, the system is executed on a single computer system, without requiring a connection to a server computer. In a further example embodiment, the system is run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Apple Inc. located in Cupertino, CA). In yet a further embodiment, the system is run on a Mac OS® environment (Mac OS is a registered trademark of Apple Inc. located in Cupertino, CA). In a further embodiment, the system is run on a Linux® environment (Linux is a registered trademark of Linus Torvalds in the United States and other countries). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

In some embodiments, a computer program is provided, and the program is embodied on a computer-readable medium and utilizes a Structured Query Language (SQL) with a client user interface front-end for administration and a web interface for standard user input and reports. In some embodiments, the system is web enabled and is run on a business entity intranet. In some embodiments, the system is fully accessed by individuals having an authorized access outside a firewall of a business-entity through the Internet. The system is flexible and designed to run in various different environments without compromising any major functionality.

As used herein, the term “database” may refer to either a body of data, a relational database management system (RDBMS), or to both. A database may include any collection of data including hierarchical databases, relational databases, flat file databases, object-relational databases, object oriented databases, and any other structured collection of records or data that is stored in a computer system. The above examples are for example only, and thus, are not intended to limit in any way the definition and/or meaning of the term database. Examples of RDBMS's include, but are not limited to including, Oracle® Database, MySQL, IBM® DB2, Microsoft® SQL Server, Sybase®, and PostgreSQL. However, any database implementation (e.g., relational, document-based) may be used that enables the system and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California).

The term processor, as used herein, may refer to central processing units, microprocessors, microcontrollers, reduced instruction set circuits (RISC), application specific integrated circuits (ASIC), logic circuits, and any other circuit or processor capable of executing the functions described herein.

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are for example only, and are thus not limiting as to the types of memory usable for storage of a computer program.

The computer-implemented methods discussed herein may include additional, less, or alternate actions, including those discussed elsewhere herein. The methods may be implemented via one or more local or remote processors, transceivers, servers, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

While the disclosure has been described in terms of various specific embodiments, those skilled in the art will recognize that the disclosure can be practiced with modification within the spirit and scope of the claims.

As used herein, the term “non-transitory computer-readable media” is intended to be representative of any tangible computer-based device implemented in any method or technology for short-term and long-term storage of information, such as, computer-readable instructions, computer-executable instructions, data structures, program modules and sub-modules, or other data in any device. Therefore, the methods described herein may be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, an element or step recited in the singular and preceded with the word “a” or “an” should be understood as not excluding plural elements or steps, unless such exclusion is explicitly recited. For example, reference to “an allocation plan” may refer to a plurality of allocation plans. Furthermore, references to “example embodiment” or “one embodiment” of the present disclosure are not intended to be interpreted as excluding the existence of additional embodiments that also incorporate the recited features.

As will be appreciated based on the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The article of manufacture containing the computer code may be made and/or used by executing the code directly from one medium, by copying the code from one medium to another medium, or by transmitting the code over a network.

In addition, although various elements of the PHP system described herein as including general processing and memory devices, it should be understood that PHP system is a specialized computer configured to perform the steps described herein for generating personalized health plans.

This written description uses examples to disclose the embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial locational differences from the literal language of the claims.

What is claimed is:

1. A personalized health plan (PHP) computing system comprising at least one processor in communication with at least one memory, wherein the at least one processor is configured to:
receive member health data associated with a member from at least one of a database or a member computing device;
apply the member health data to an observation model, wherein the observation model is trained to identify health conditions of the member based upon historical health data;
receive a health conditions output from the observation model, the health conditions output including one or more health conditions associated with the member based upon the member health data;
apply the health conditions output to one or more service models, wherein the one or more service models are trained to generate treatment plans associated with care for different health conditions;
receive a service response output from the service model, the service response output including a recommended treatment plan comprising a recommended health appointment for the member based upon a health condition of the one or more health conditions;
cause a first notification for the recommended health appointment to be transmitted to a care provider computing device associated with a care provider to conduct the recommended health appointment for the member; and
cause a second notification for the recommended health appointment to be transmitted to the member computing device to notify the member of the recommended health appointment with the care provider.

2. The PHP computing system of claim 1, wherein the one or more service models are further trained to prioritize health conditions based upon historical severities of the health conditions.

3. The PHP computing system of claim 2, wherein the one or more health conditions comprise a plurality of health conditions, wherein the service response output further comprises severities of the plurality of health conditions, and wherein the at least one processor is further configured to cause the first notification and the second notification to be transmitted before other notifications associated with the plurality of health conditions based upon the health condition being a highest priority health condition.

4. The PHP computing system of claim 3, wherein the at least one processor is further configured to cause a third notification to be transmitted to the member computing device after causing the first notification and the second notification to be transmitted, the third notification associated with a next highest priority health condition of the plurality of health conditions and comprising a recommended treatment to address the next highest priority health condition.

5. The PHP computing system of claim 1, wherein the at least one processor is further configured to:
receive updated member health data associated with the member from at least one of the database or the member computing device, the updated member health data including at least one update to the member health data;
input the updated member health data to the observation model
receive an updated health conditions output from the observation model;
input the updated health conditions output to the one or more service models; and
receive an updated service response output from the one or more service models, the updated service response output comprising an updated recommended treatment plan including a plurality of treatment actions, each treatment action being assigned a priority level.

6. The PHP computing system of claim 1, wherein the at least one processor is further configured to generate a graphical user interface (GUI), the GUI comprising:
a first selector indicating that the recommended health appointment has been recommended to the care provider based upon the first notification; and
one or more second selectors associated with one or more additional appointments assigned to the care provider, wherein the GUI is updated in real time based upon outputs associated with the care provider from the one or more service models.

7. The PHP computing system of claim 1, wherein the health conditions output comprises duplicates of at least one health condition that were identified based upon different factors in the member health data, and wherein the at least one processor is further configured to:
apply the member health data to an assurance model, the assurance model trained to de-duplicate health data based upon historical duplicates identified in the historical health data; and
receive an assurance output from the assurance model, the assurance output comprising an updated health conditions output including no duplicates of the at least one health condition, wherein the health conditions output is updated with the updated health conditions output before the at least one processor applies the health conditions output to the one or more service models.

8. At least one non-transitory computer-readable storage medium with instructions stored thereon for personal health planning, wherein the instructions, when executed by at least one processor, cause the at least one processor to:
receive member health data associated with a member from at least one of a database or a member computing device;
apply the member health data to an observation model, wherein the observation model is trained to identify health conditions of the member based upon historical health data;
receive a health conditions output from the observation model, the health conditions output including one or more health conditions associated with the member based upon the member health data;
apply the health conditions output to one or more service models, wherein the one or more service models are trained to generate treatment plans associated with care for different health conditions;
receive a service response output from the service model, the service response output including a recommended treatment plan comprising a recommended health appointment for the member based upon a health condition of the one or more health conditions;
cause a first notification for the recommended health appointment to be transmitted to a care provider computing device associated with a care provider to conduct the recommended health appointment for the member; and
cause a second notification for the recommended health appointment to be transmitted to the member computing device to notify the member of the recommended health appointment with the care provider.

9. The at least one non-transitory computer-readable storage medium of claim 8, wherein the one or more service models are further trained to prioritize health conditions based upon historical severities of the health conditions.

10. The at least one non-transitory computer-readable storage medium of claim 9, wherein the one or more health conditions comprise a plurality of health conditions, wherein the service response output further comprises severities of the plurality of health conditions, and wherein the instructions further cause the at least one processor to cause the first notification and the second notification to be transmitted before other notifications associated with the plurality of health conditions based upon the health condition being a highest priority health condition.

11. The at least one non-transitory computer-readable storage medium of claim 10, wherein the instructions further cause the at least one processor to cause a third notification to be transmitted to the member computing device after causing the first notification and the second notification to be transmitted, the third notification associated with a next highest priority health condition of the plurality of health conditions and comprising a recommended treatment to address the next highest priority health condition.

12. The at least one non-transitory computer-readable storage medium of claim 8, wherein the instructions further cause the at least one processor to:
receive updated member health data associated with the member from at least one of the database or the member computing device, the updated member health data including at least one update to the member health data;
input the updated member health data to the observation model
receive an updated health conditions output from the observation model;
input the updated health conditions output to the one or more service models; and
receive an updated service response output from the one or more service models, the updated service response output comprising an updated recommended treatment plan including a plurality of treatment actions, each treatment action being assigned a priority level.

13. The at least one non-transitory computer-readable storage medium of claim 8, wherein the instructions further cause the at least one processor to generate a graphical user interface (GUI), the GUI comprising:
a first selector indicating that the recommended health appointment has been recommended to the care provider based upon the first notification; and
one or more second selectors associated with one or more additional appointments assigned to the care provider, wherein the GUI is updated in real time based upon outputs associated with the care provider from the one or more service models.

14. The at least one non-transitory computer-readable storage medium of claim 8, wherein the health conditions output comprises duplicates of at least one health condition that were identified based upon different factors in the member health data, and wherein the instructions further cause the at least one processor to:
apply the member health data to an assurance model, the assurance model trained to de-duplicate health data based upon historical duplicates identified in the historical health data; and
receive an assurance output from the assurance model, the assurance output comprising an updated health conditions output including no duplicates of the at least one health condition, wherein the health conditions output is updated with the updated health conditions output before the at least one processor applies the health conditions output to the one or more service models.

15. A computer-implemented method for personalized health planning, the computer-implemented method implemented by at least one processor and at least one memory, the computer-implemented method comprising:
receiving member health data associated with a member from at least one of a database or a member computing device;
applying the member health data to an observation model, wherein the observation model is trained to identify health conditions of the member based upon historical health data;
receiving a health conditions output from the observation model, the health conditions output including one or more health conditions associated with the member based upon the member health data;
applying the health conditions output to one or more service models, wherein the one or more service models are trained to generate treatment plans associated with care for different health conditions;
receiving a service response output from the service model, the service response output including a recommended treatment plan comprising a recommended health appointment for the member based upon a health condition of the one or more health conditions;
causing a first notification for the recommended health appointment to be transmitted to a care provider computing device associated with a care provider to conduct the recommended health appointment for the member; and
causing a second notification for the recommended health appointment to be transmitted to the member computing device to notify the member of the recommended health appointment with the care provider.

16. The computer-implemented method of claim 15, wherein the one or more service models are further trained to prioritize health conditions based upon historical severities of the health conditions.

17. The computer-implemented method of claim 16, wherein the one or more health conditions comprise a plurality of health conditions, wherein the service response output further comprises severities of the plurality of health conditions, and wherein the computer-implemented method further comprises:
causing the first notification and the second notification to be transmitted before other notifications associated with the plurality of health conditions based upon the health condition being a highest priority health condition; and
causing a third notification to be transmitted to the member computing device after causing the first notification and the second notification to be transmitted, the third notification associated with a next highest priority health condition of the plurality of health conditions and comprising a recommended treatment to address the next highest priority health condition.

18. The computer-implemented method of claim 15, further comprising:
receiving updated member health data associated with the member from at least one of the database or the member computing device, the updated member health data including at least one update to the member health data;
inputting the updated member health data to the observation model
receiving an updated health conditions output from the observation model;
inputting the updated health conditions output to the one or more service models; and
receiving an updated service response output from the one or more service models, the updated service response output comprising an updated recommended treatment plan including a plurality of treatment actions, each treatment action being assigned a priority level.

19. The computer-implemented method of claim 15, further comprising generating a graphical user interface (GUI), the GUI comprising:
a first selector indicating that the recommended health appointment has been recommended to the care provider based upon the first notification; and
one or more second selectors associated with one or more additional appointments assigned to the care provider, wherein the GUI is updated in real time based upon outputs associated with the care provider from the one or more service models.

20. The computer-implemented method of claim 15, wherein the health conditions output comprises duplicates of at least one health condition that were identified based upon different factors in the member health data, and wherein the computer-implemented method further comprises:
applying the member health data to an assurance model, the assurance model trained to de-duplicate health data based upon historical duplicates identified in the historical health data; and
receiving an assurance output from the assurance model, the assurance output comprising an updated health conditions output including no duplicates of the at least one health condition, wherein the health conditions output is updated with the updated health conditions output before the at least one processor applies the health conditions output to the one or more service models.
